(19) United States
(12) Patent Application Publication
(10) Pub. No.: US 2015/0276614 A1
(43) Pub. Date: Oct. 1, 2015

(54) SMOKE CONCENTRATION MEASUREMENT SYSTEM AND RELATED METHODS

(71) Applicant: Factory Mutual Insurance Company, Johnston, RI (US)

(72) Inventors: Benjamin D. DITCH, Shrewsbury, MA (US); Marcos Chaos, Boston, MA (US); Myles Silva, Chepachet, RI (US); Sai K. Thumuluru, Quincy, MA (US)

(73) Assignee: Factory Mutual Insurance Company, Johnston, RI (US)

(21) Appl. No.: 14/230,398

(22) Filed: Mar. 31, 2014

(51) Int. Cl. G01N 21/85; G01N 33/00; G01N 21/39

(52) U.S. Cl. CPC G01N 21/85; G01N 21/39; G01N 33/0027; G01N 2201/06113; G01N 2021/8578

(57) ABSTRACT

A smoke concentration measurement system includes: a beam extension chamber having a body defining a smoke channel having a longitudinal axis, the smoke channel having a smoke inlet and a smoke outlet at opposite ends of the longitudinal axis, a first reflective surface located on a first lateral side of the longitudinal axis, an entrance window located on the first lateral side of the longitudinal axis, a second reflective surface located on a second lateral side of the longitudinal axis, and an exit window located on the second lateral side of the longitudinal axis. The system also includes a laser light source adapted to emit laser light onto the entrance window, the laser light reflecting back and forth between the first reflective surface and the second reflective surface toward the exit window, and a first light measurement device adapted to receive laser light exiting the exit window. Other features, as well as a method of measuring the concentration of smoke, are also described.

### TECHNICAL FIELD

This patent application relates generally to methods and systems for testing properties of smoke. More specifically, this patent application relates to methods and systems for measuring the concentration of smoke.

### BACKGROUND

Methods and systems for measuring the concentration of smoke are known. Some known techniques pass a laser through a stream of the smoke, and measure the amount of laser light extinction caused by the smoke. The amount of laser light extinction can then be correlated to the concentration of the smoke.

Known techniques typically suffer from low sensitivity at low smoke concentrations. In order to compensate for low sensitivity, traditional systems have extended the path length of the laser beam. However, extending the path length results in an area averaged measurement of the smoke concentration, which typically requires an assumption that the smoke concentration is uniform along the path length.

### SUMMARY

According to an embodiment, the invention provides a smoke concentration measurement system, comprising: a beam extension chamber comprising: a body defining a smoke channel having a longitudinal axis, the smoke channel having a smoke inlet and a smoke outlet at opposite ends of the longitudinal axis, a first reflective surface located on a first lateral side of the longitudinal axis, an entrance window located on the first lateral side of the longitudinal axis, a second reflective surface located on a second lateral side of the longitudinal axis, and an exit window located on the second lateral side of the longitudinal axis; a laser light source adapted to emit laser light onto the entrance window, the laser light reflecting back and forth between the first reflective surface and the second reflective surface toward the exit window; and a first light measurement device adapted to receive laser light exiting the exit window. According to embodiments, the system can further comprise a smoke collection element, such as a gravimetric filter, in fluid communication with the smoke channel, wherein the smoke collection element is located downstream from the beam extension chamber.

According to an embodiment, the invention provides a method of measuring the concentration of smoke, comprising: directing a flow of smoke through a smoke channel from a smoke inlet to a smoke outlet, wherein the smoke channel defines a longitudinal axis between the smoke inlet and the smoke outlet; projecting laser light into the smoke channel through an entrance window; deflecting the laser light back-and-forth across the longitudinal axis from the entrance window to an exit window; and measuring the intensity of laser light exiting the exit window. According to embodiments, the method can further include measuring the concentration of the smoke by gravimetric filtering.

### BRIEF DESCRIPTION OF THE DRAWINGS

The foregoing and other features and advantages will be apparent from the following, more particular, description of various exemplary embodiments, as illustrated in the accompanying drawings, wherein like reference numbers generally indicate identical, functionally similar, and/or structurally similar elements.

FIG. 1 is top view of an embodiment of a smoke concentration measurement system.

FIG. 2 is a side perspective view of an embodiment of a beam extension chamber of FIG. 1.

FIG. 3A is a side view of an embodiment of a body of the beam extension chamber of FIG. 2.

FIG. 3B is a cross-sectional view of the body of FIG. 3A, taken along line 3B-3B.

FIG. 4A is a side view of an alternative embodiment of a body.

FIG. 4B is a partial cross-sectional view of the body of FIG. 4A, taken along line 4B-4B.

FIG. 4C is an end view of the body of FIG. 4A.

FIG. 5A is a top view of an embodiment of a lateral adjustment bracket for use with the body of FIG. 4A.

FIG. 5B is a side view of the lateral adjustment bracket of FIG. 5A.

### DETAILED DESCRIPTION

Various embodiments of the invention are discussed in detail below. While specific embodiments are discussed, it should be understood that this is done for illustration purposes only. A person skilled in the relevant art will recognize that other components and configurations can be used without departing from the spirit and scope of the invention.

Embodiments of the smoke concentration system described herein can provide an approximately point source measurement of smoke at low concentrations. For example, the system can be implemented to evaluate the performance of smoke detectors in large data centers, however, other applications are possible.

Conventional measurement of smoke concentration using laser light extinction tend to be limited by the low-end sensitivity of the system, which may be directly related to the optical path length of the system. Some conventional systems have addressed this problem by extending the laser beam across a long path length in the smoke flow. However, this typically results in an area averaged measurement of smoke concentration, which requires an assumption that the smoke concentration is uniform along the optical path length. Embodiments of the system described herein can remove or reduce the need for a long path length in the smoke flow, thereby allowing for an approximately point source measurement of the smoke concentration; thereby substantially nullifying any assumption of the distribution of smoke concentration along the laser path length.

Embodiments of the system described herein can also provide a secondary measurement of smoke concentration, e.g., to validate the laser light extinction based measurement. The secondary measurement can be acquired using filters (e.g., gravimetric filters) located in the smoke flow, as will be described in more detail below. The secondary measurement can provide increased accuracy over a system that only utilizes a laser light extinction based measurement, for example, due to assumptions that may be required to convert measurements from the laser system (e.g., photodiode voltages) to smoke concentration. According to embodiments, the gravimetric smoke measurements represent a time-averaged smoke concentration over the entire collection period. Comparison of the total smoke measured by the laser system and the gravimetric filter, both over the same time period, can provide a validation of the assumptions required to calculate smoke concentration from the laser system. Embodiments of the invention can provide a compact and portable configuration of a multi-pass laser extinction measurement system, having increased low-end sensitivity and adjustability compared to conventional systems.

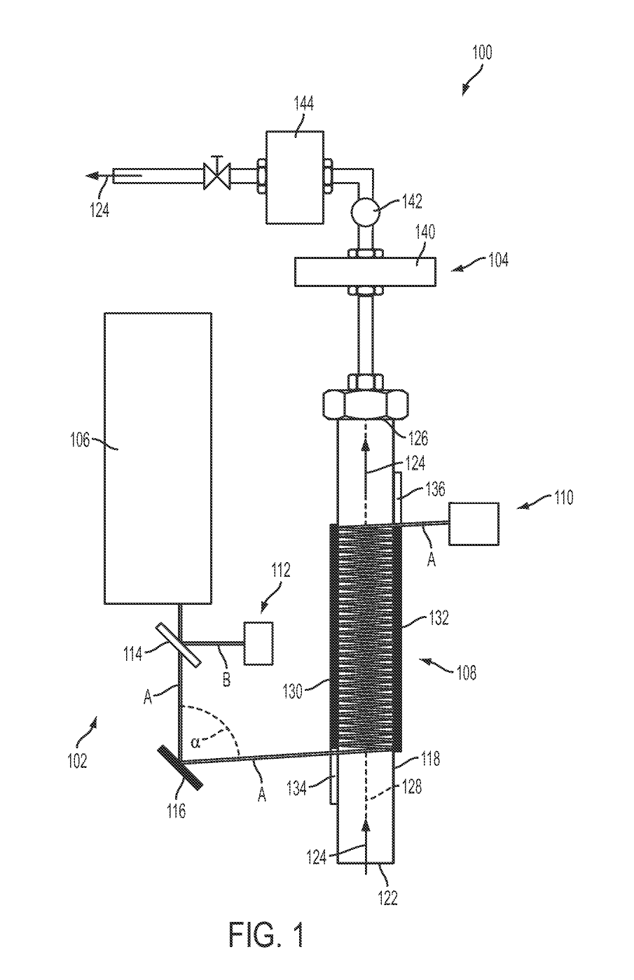

Referring to FIG. 1, embodiments of the smoke concentration measurement system 100 can include a laser-light-based meter 102, as well as a gravimetric-based meter 104. The laser-light-based meter 102 can include a laser light source 106, such as a 1.2 mW stabilized Helium-Neon (HeNe) laser, however, other embodiments are possible. According to embodiments, the laser light source 106 can operate at a wavelength of between about 4×10⁷ m and 8×10⁷ m, e.g., about 6.328×10⁷ m, however, other embodiments are possible. The laser-light-based meter 102 can also include a beam extension chamber 108 through which sample smoke flows, a first light measurement device 110, and a second light measurement device 112. According to embodiments, the first and/or second light measurement devices 110, 112 can comprise silicon photodiodes, however, other embodiments are possible. Although not shown, control system such as a computer, PLC, or other similar device, can receive and process output signals from the first and second light measurement devices 110.

Still referring to FIG. 1, the laser-light-based meter 102 can further include a beam splitter 114, such as a non-polarizing beam splitter, that divides laser light output from the laser light source 106 into a measurement beam A and a reference beam B, as will be described in more detail, below. An adjustable mirror 116, such as a dielectric mirror, can be provided between the beam splitter 114 and the beam extension chamber 108. According to embodiments, the mirror 116 can be located on an adjustable mount that provides for adjustment of the mirror's angle a. By adjusting the angle a, the angle at which the measurement beam A contacts the beam extension chamber 108 can be adjusted.

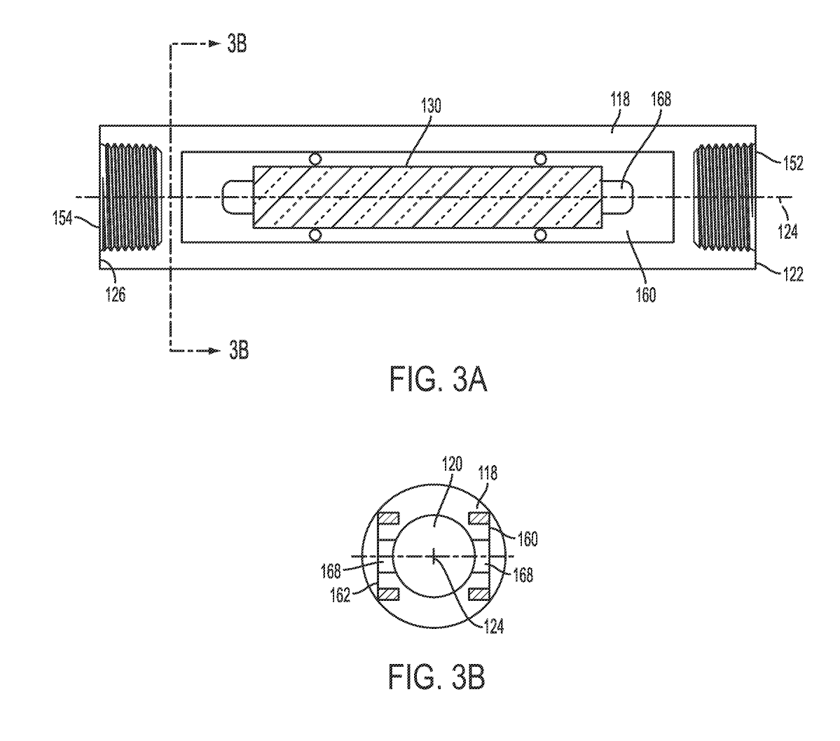

Still referring to FIG. 1, the beam extension chamber 108 can include a body 118 defining a smoke channel 120 (see FIG. 3B) defining an inlet 122 that receives gas flow 124 from a test enclosure, e.g., a test fire. A vacuum pump or other structure can be provided to draw the gas flow 124 through the beam extension chamber 108 and other components. The smoke channel 120 also defines an outlet 126 through which the gas flow 124 exits the smoke channel 120. Embodiments of the beam extension chamber 108 can define a longitudinal axis 128 between the inlet 122 and outlet 126, as shown in FIG. 1.

Still referring to FIG. 1, the beam extension chamber 108 can include first and second reflective surfaces 130, 132 located on opposite sides of the longitudinal axis 128. According to embodiments, the reflective surfaces 130, 132 can comprise dielectric mirrors mounted to the body 118, however, other configurations are possible. The beam extension chamber 108 can also include an entrance window 134 located upstream of the first reflective surface 130, and an exit window 136 located downstream of the second reflective surface 132. According to embodiments, the entrance and exit windows 134, 136 can comprise substantially transparent substrates, such as AR-coated broadband precision windows mounted to the body 118, however, other configurations are possible.

The gravimetric-based meter 104 can include a smoke collection element 140, such as a filter housing, located downstream of the beam extension chamber 108 to collect smoke samples. According to embodiments, the smoke collection element can removably house a filter or other collection medium. According to embodiments, the filter can comprise a 2 micron quartz filter, however, other embodiments are possible. A pressure gauge 142, such as a 0-20 psia pressure transducer, can be provided in the gas flow 124 downstream of the extension chamber 108. Additionally or alternatively, one or more flow gauges 144, such as a 0-100 L/min mass flow meter, can be provided in the gas flow 124 downstream of the beam extension chamber 108. The pressure gauge 142 and/or flow gauge(s) 144 can measure and optionally record the flow rate and pressure of the gas flow 124. Additionally or alternatively, the pressure gauge 142 and/or flow gauge(s) 144 can provide feedback to the vacuum pump in case adjustments in the gas flow 124 are necessary.

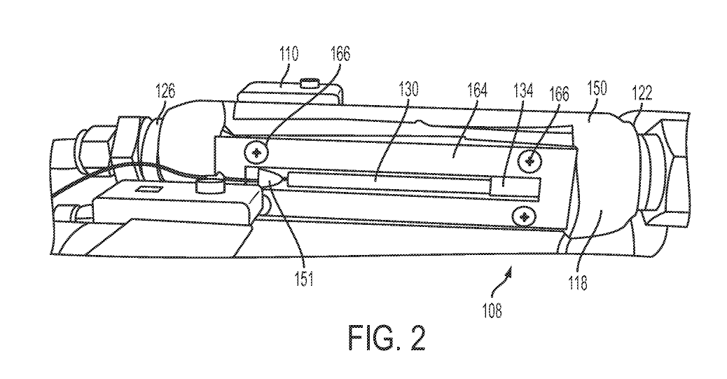

Referring to FIG. 2, a heating element 150 can be coupled to the body 118 of the beam extension chamber 108. For example, the heating element 150 can comprise heat tape provided around all or a portion of the body 118. To reduce thermophoretic smoke deposition on optical surfaces of the beam extension chamber 108, the heating element 150 can maintain the chamber 108 at or above the temperature of the sample gas, e.g., at about 20° C. higher than the sample gas temperature. A controller (not shown) including a temperature gauge 151 can adjust and maintain the temperature of the heating element 150.

Referring to FIGS. 1 and 2, laser light exiting the laser light source 106 is split using the beam splitter 114 into the measurement beam A and the reference beam B. The reference beam B is directed by the beam splitter 114 onto the second light measurement device 112. Light intensity measured by the second light measurement device 112 can be used as a reference measurement of the light intensity exiting the laser light source 106. The beam splitter 114 directs the measurement beam A onto the adjustable mirror 116, which in turn directs the measurement beam onto the beam extension chamber 108, where the beam can enter the smoke channel 120 through entrance window 134.

As shown in FIG. 1, once the measurement beam A enters the smoke channel 120, the beam is reflected back and forth across the smoke channel 120 by the first and second reflective surfaces 130, 132, thereby extending the optical path length of the beam. Once the beam reaches the exit window 134, it exits the smoke channel 120 and is received by the first light measurement device 110, as shown in FIG. 2. A controller, such as a computer, PLC, or other device can measure the attenuation of the beam, e.g., by comparing the light intensity measured by the first and second light measurement devices 110, 112. The degree of attenuation can then be correlated to a concentration of smoke following through the smoke channel 120 between the entrance window 134 and the exit window 136.

The angle at which the measurement beam A enters the smoke channel 120 can be varied by changing the angle a of the adjustable mirror 116. This adjustment in turn determines the number of passes (mirror reflections) the beam makes between the entrance window 134 and exit window 136, and therefore determines the overall path length within the smoke channel 120. According to an embodiment, the optical path length can be calculated based on the number of reflection points on each reflective surface, the perpendicular distance separating the reflective surfaces, and the angle of the beam between the reflective surfaces.

According to an embodiment, the smoke channel 120 can define a length (e.g., between smoke inlet 122 and smoke outlet 126) of between about 2 inches and about 8 inches, for example, between about 4 inches and about 6 inches, and the measurement beam can make between about 15 and 40 passes between the entrance window and the exit window. According to such embodiments, the optical path length (L) can be between about 2 feet and about 8 feet, for example, between about 4 feet and about 6 feet. According to an embodiment having a separation distance (S) of 1.36 inches and a number of reflection points per mirror (N) of 21, the optical path length L is about 4.85 feet. One of ordinary skill will appreciate from this disclosure, however, that other dimensions and configurations than those described above are possible.

Referring to FIGS. 3A and 3B, an embodiment of body 118 is shown. In FIG. 3A, the first reflective surface 130, in the form of a mirror, is also shown. The body 118 can be constructed of a metal tube, such as steel, having an outer diameter of between one and three inches, e.g., about 1.75 inches, and an inner diameter of between 0.5 and 2 inches, e.g., about 1 inch. One of ordinary skill in the art will understand, however, that other dimensions are possible. The smoke channel 120 can extend longitudinally through the body 118. Coupling devices, such as threads 152, 154 or other structures can be provided to facilitate attachment to upstream and downstream components.

Opposite sides of the body 118 can include mounting surfaces 160, 162 for the reflective surfaces 130, 132 and/or windows 134, 136. For example, with reference to FIG. 2, one or more mounting brackets 164 (only one shown) can secure the reflective surface 130 and entrance window 134 in place on the body 118, e.g., using machine bolts 166 or other fasteners. Although not shown, a similar arrangement can be used for the reflective surface 132 and exit window 136. Referring to FIG. 3B, longitudinal slots 168 can be provided in body 118 to provide an optical pathway for the measurement beam between the reflective surfaces 130, 132. The slots 168 can be in registry with at least a portion of the reflective surfaces 130, 132 and windows 134, 136. According to embodiments, each slot can have a length of between about 3 inches and 6 inches (e.g., about 5 inches) and width of between about 0.24 inches and 0.75 inches (e.g., about 0.4 inches), however, other sizes are possible. Adjustment slots in the mounting holes for the brackets 164 (see FIG. 2), or other similar structures, can be provided to allow adjustment of the reflective surfaces 130, 132 and windows 134, 136 along the longitudinal axis 124. This allows the position of reflective surfaces 130, 132 and windows 134, 136 to be adjusted in response to changes in the angle a of the adjustable mirror 116, and resulting changes in the number of reflections per mirror (N) and location of the measurement beam's entrance point and exit point from the smoke channel 120.

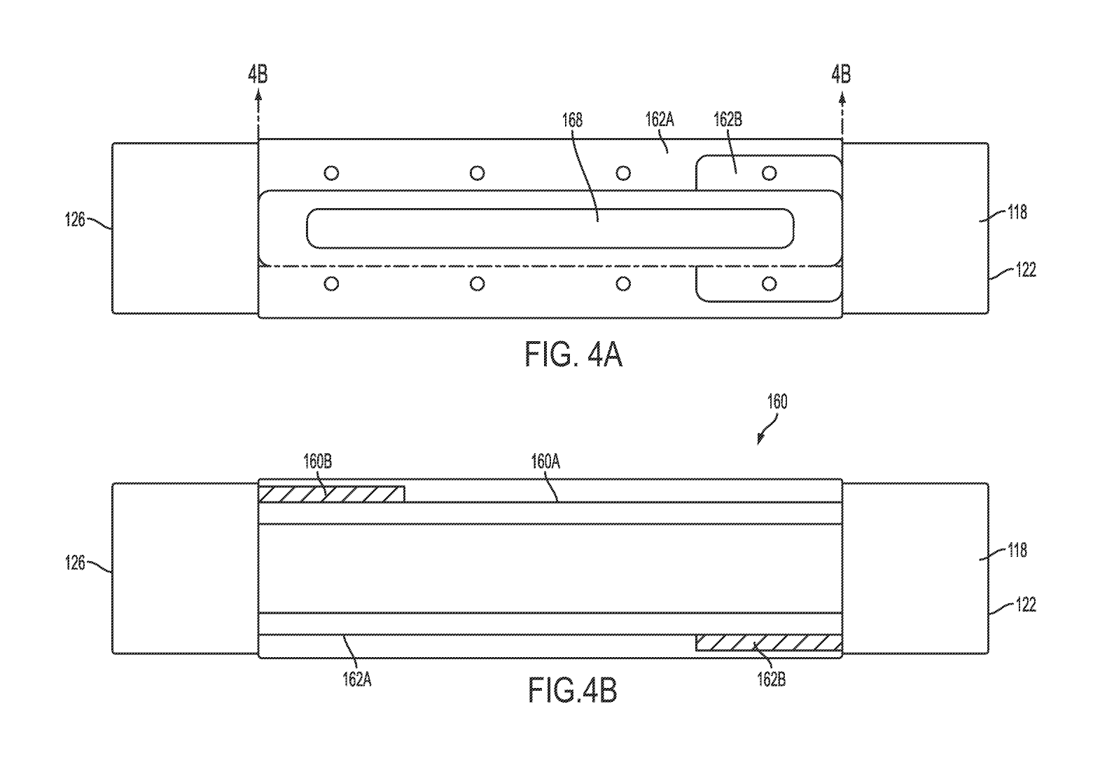

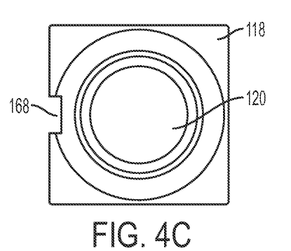

Referring to FIGS. 4A-4C, another embodiment of the body 118 is shown. Body 118 is substantially similar to the version described in connection with FIGS. 3A, 3B, with differences described herein below. Referring to FIGS. 4A and 4B, the mounting surfaces 160, 162 can each be divided into multi-planar portions to allow for independent mounting and adjustment of the reflective surfaces 130, 132 and windows 134, 136, respectively. For example, as shown in FIG. 4B, the mounting surface 160 can be divided into a primary surface 160A for mounting the first reflective surface 130, and a secondary surface 160B, located on a different plane, for mounting the entrance window 134. The secondary surface 160B can have a larger area than the primary surface 160A, as shown, however, other variations are possible. The mounting surface 162 can have a similar configuration to surface 160, as shown in FIG. 4B. The windows 134, 136 and reflective surfaces 130, 132 can include slotted mounting holes to allow for longitudinal adjustment, as described previously in connection with FIGS. 3A and 3B.

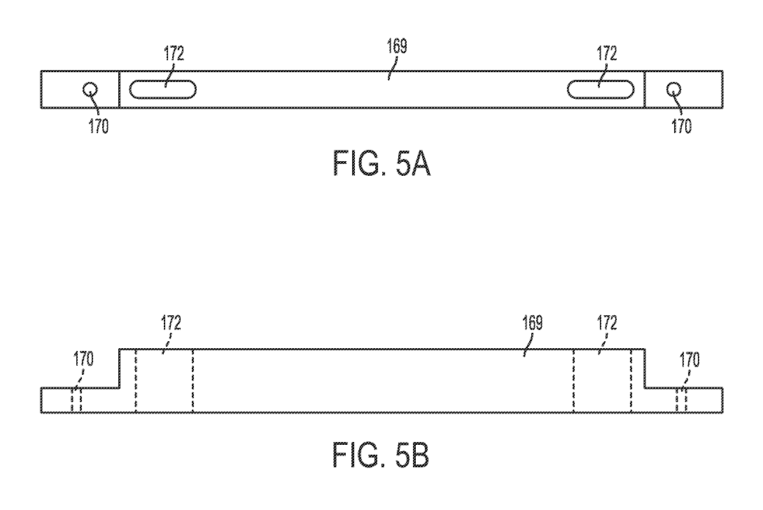

As shown in FIG. 4C, the body 118 can include a longitudinal slot 168 extending along all or a part of its lower surface. The longitudinal slot 168 can be dimensioned to receive a lateral adjustment bracket 169, shown in FIGS. 5A and 5B. The lateral adjustment bracket 169 can include mounting holes 170 for securing the bracket 169 to a work surface. The bracket 169 can also include slotted holes 172 for securing the bracket 169 to the body 118, e.g., using screws extending into holes (not shown) in longitudinal slot 168. The slotted holes 172 can allow the position of the body 118 to be adjusted along the length of adjustment bracket 169, e.g., for accurate positioning of the body 118 with respect to the measurement beam. As shown in FIGS. 4A and 4B, the ends of body 118 can be round to receive heating elements on either end of the beam extension chamber 108. The heating elements can be wired in series to a controller with a thermocouple mechanically fastened to the top of the chamber, to control the temperature of the beam extension chamber 108.

While various embodiments of the present invention have been described above, it should be understood that they have been presented by way of example only, and not limitation. For example, the invention can be applied to the measurement of many other particulates in an air stream and is not limited to the measurement of smoke. Thus, the breadth and scope of the present invention should not be limited by any of the above-described embodiments, but should instead be defined only in accordance with the following claims and their equivalents.

What is claimed:

1. A smoke concentration measurement system, comprising:
a beam extension chamber comprising:
a body defining a smoke channel having a longitudinal axis, the smoke channel having a smoke inlet and a smoke outlet at opposite ends of the longitudinal axis,
a first reflective surface located on a first lateral side of the longitudinal axis,
an entrance window located on the first lateral side of the longitudinal axis,
a second reflective surface located on a second lateral side of the longitudinal axis, and
an exit window located on the second lateral side of the longitudinal axis;
a laser light source adapted to emit laser light onto the entrance window, the laser light reflecting back and forth between the first reflective surface and the second reflective surface toward the exit window; and
a first light measurement device adapted to receive laser light exiting the exit window.

2. The smoke concentration measurement system of claim 1, further comprising:
a beam splitter located upstream of the beam extension chamber, the beam splitter adapted to split the laser light into a measurement beam directed onto the entrance window, and a reference beam; and
a second light measurement device adapted to receive the reference beam.

3. The smoke concentration measurement system of claim 2, further comprising a control system in communication with the first light measurement device and the second light measurement device, the control system programmed to calculate smoke concentration based at least in part on intensity of the laser light received by the first light measurement device, and intensity of the reference beam.

4. The smoke concentration measurement system of claim 1, further comprising a smoke collection element in fluid communication with the smoke channel, the smoke collection element located downstream from the beam extension chamber.

5. The smoke concentration measurement system of claim 4, wherein the smoke collection element comprises a gravimetric filter.

6. The smoke concentration measurement system of claim 1, further comprising a pump adapted to draw a flow of smoke through the smoke channel.

7. The smoke concentration measurement system of claim 1, wherein at least one of the entrance and exit windows is adjustable in position along the longitudinal axis.

8. The smoke concentration measurement system of claim 1, wherein at least one of the first reflective surface and second reflective surfaces is adjustable in position along the longitudinal axis.

9. The smoke concentration measurement system of claim 1, wherein at least one of the first and second reflective surfaces comprises a mirror.

10. The smoke concentration measurement system of claim 1, wherein at least one of the first and second windows comprises a substantially transparent substrate.

11. The smoke concentration measurement system of claim 1, further comprising an adjustable mirror located between the laser light source and the beam extension chamber, the adjustable mirror adapted to adjust the angle with which the light laser light contacts the entrance window.

12. The smoke concentration measurement system of claim 1, wherein the entrance window is located upstream with respect to the exit window.

13. The smoke concentration measurement system of claim 1, wherein the first light measurement device comprises a photodiode detector.

14. The smoke concentration measurement system of claim 2, wherein the second light measurement device comprises a photodiode detector.

15. The smoke concentration measurement system of claim 1, wherein the beam extension chamber defines a first longitudinal slot and a second longitudinal slot, the first and second longitudinal slots being substantially in registry with the first and second reflective surfaces.

16. The smoke concentration measurement system of claim 1, further comprising a heat element coupled to the beam extension chamber.

17. The smoke concentration measurement system of claim 16, wherein the heat element comprises heat tape.

18. The smoke concentration measurement system of claim 1, wherein the smoke channel defines a length of between about 2 inches and about 8 inches, and the laser light defines an optical path between the entrance window and the exit window of between about 2 feet and about 8 feet.

19. The smoke concentration measurement system of claim 18, wherein the length of the smoke channel is between about 4 inches and about 6 inches, and the optical path is between about 4 feet and about 6 feet.

20. A method of measuring the concentration of smoke, comprising:
directing a flow of smoke through a smoke channel from a smoke inlet to a smoke outlet, wherein the smoke channel defines a longitudinal axis between the smoke inlet and the smoke outlet;
projecting laser light into the smoke channel through an entrance window;
deflecting the laser light back-and-forth across the longitudinal axis from the entrance window to an exit window; and
measuring the intensity of laser light exiting the exit window.

21. The method of claim 20, further comprising:
splitting the laser light into a measurement beam and a reference beam;
directing the measurement beam into the entrance window; and
measuring the intensity of the reference beam.

22. The method of claim 21, further comprising determining the concentration of smoke based on the intensity of laser light exiting the exit window, and the intensity of the reference beam.

23. The method of claim 20, further comprising:
measuring the concentration of the smoke by gravimetric filtering.

24. The method of claim 20, further comprising:
varying the amount of passes the laser light makes between the entrance window and the exit window by adjusting the angle at which the laser light contacts the entrance window.

25. The method of claim 24, wherein the smoke channel defines a length of between about 2 inches and about 8 inches, and the laser light makes between about 15 and 40 passes between the entrance window and the exit window.

26. The smoke concentration measurement system of claim 3, wherein the control system is further programmed to calculate smoke concentration based on a rate of gas flow through the smoke channel.

27. The method of claim 20, further comprising:
measuring the flow of smoke passing through the smoke channel.
